(19) United States
(12) Patent Application Publication
(10) Pub. No.: US 2009/0131963 A1
(43) Pub. Date: May 21, 2009

(54) Scalpel blade protector

(76) Inventor: James Edwin Rasco, The Woodlands, TX (US)

Correspondence Address: G. TURNER MOLLER, JR., 711 NORTH CARANCAHUA, SUITE 720, CORPUS CHRISTI, TX, 78475, US

(21) Appl. No.: 12/319,944

(22) Filed: Jan. 15, 2009

Related U.S. Application Data
(63) Continuation-in-part of application No. 11/226,753, filed on Sep. 14, 2005.

(51) Int. Cl. A61B 17/3211

(52) U.S. Cl. 606/172

(57) ABSTRACT

A scalpel blade protector comprises an inner housing friction fit onto a scalpel handle and an outer housing or blade guard slidably mounted on the inner housing for movement between positions exposing and shielding the scalpel blade. The blade protector is attached to conventional scalpel handles. Some embodiments show a blade protector for a flat scalpel handle. Some embodiments show a blade protector for a round or hexagonal scalpel handle. The blade guard and inner housing include a latch mechanism so the blade guard is latched in the position exposing the scalpel blade and in the position shielding the scalpel blade.

This application is a continuation-in-part of application Ser. No. 11/226,753, filed Sep. 14, 2005.

This invention relates to a blade protector for a surgeon's scalpel and more particularly to a blade protector for a conventional scalpel.

### BACKGROUND OF THE INVENTION

A movement exists to encourage or require surgeon's scalpels to have blade protectors in an attempt to minimize accidental cuts during surgery. The broad concept is to provide a shield or guard that can be moved by the surgeon from a stowed position exposing the blade to an operative position covering the blade. Not surprisingly, there have been proposed a series of specially designed scalpels that incorporate blade protectors such as found in U.S. Pat. Nos. 3,906,626; 4,414,974; 4,576,164; 4,735,202; 5,292,329; 5,309,641; 5,330,492; 5,417,704; 5,423,843; 5,620,454; 5,868,771 and 6,626,925 as well as printed patent application 2004/0087989.

### SUMMARY OF THE INVENTION

In this invention, it is recognized that many surgeons have become accustomed to conventional scalpel handles and are somewhat uncomfortable with handles that are specifically modified to provide movable blade guards. Accordingly, the scalpel guard of this invention is designed to fit onto conventional scalpel handles. Two different types of blade guards are disclosed: one is a three piece device and one is a two piece device. Because most conventional scalpel handles are either flat or round/hexagonal, two different embodiments of each type are illustrated.

In both embodiments of the three piece devices, an inner housing is fixed to the scalpel handle in a suitable manner, such as with a friction fit, a removable adhesive, shrink fit or the like. In the event the inner housing is slightly loose on the scalpel handle, a wedge or other suitable member may be used to secure the inner housing to the handle. A blade guard is mounted on the inner housing for movement between an operative extended position shielding the scalpel blade and a stowed or retracted position exposing the blade. The blade guard includes a shoulder or protuberance so the surgeon can readily push or pull the blade guard to a desired position. An outer housing covers the blade guard and is attached in any suitable manner to the inner housing so the blade guard may readily slide between the inner and outer housings.

In both embodiments of the two piece device, an inner housing provides a smooth inner surface free of mechanical latches and is frictionally fit onto the scalpel handle. A blade guard is mounted on the inner housing for movement between an operative extended position shielding the scalpel blade and a stowed or retracted position exposing the blade. The blade guard and inner housing cooperate to provide a latch mechanism latching the blade guard in the extended position and in the retracted position.

It is an object of this invention to provide an improved scalpel blade protector.

A further object of this invention is to provide an improved scalpel blade protector that may be positioned on a conventional scalpel without modifying the scalpel.

A more specific object of this invention is to provide a scalpel blade protector including an inner housing affixed to the scalpel handle and a blade guard movable between positions exposing and shielding the blade and an outer housing covering much of the inner housing and blade guard.

These and other objects and advantages of this invention will become more apparent as this description proceeds, reference being made to the accompanying drawings and appended claims.

### BRIEF DESCRIPTION OF THE DRAWINGS

FIG. 1 is a partial isometric view of a conventional scalpel handle showing the blade attachment end;

FIG. 2 is an isometric view, similar to FIG. 1, showing the scalpel handle with a blade having the cutting edge up;

FIG. 3 is an end view of the scalpel of FIGS. 1 and 2;

FIGS. 4 and 5 are isometric and cross-sectional views of the scalpel of FIGS. 2 and 3 to which has been added an inner housing;

FIGS. 6 and 7 are isometric and end views of the scalpel and inner housing of FIGS. 4 and 5 to which has been added a slidable blade guard;

FIGS. 8 and 9 are isometric and end views of the scalpel, inner housing and blade guard of FIGS. 6 and 7 to which has been added an outer housing, illustrating the outer housing in an extended or shielding position over the scalpel blade;

FIG. 10 is an isometric view similar to FIG. 8 showing the blade guard in a retracted position exposing the scalpel blade;

FIG. 11 is an isometric view of another embodiment of a conventional scalpel showing the scalpel with the cutting edge down;

FIG. 12 is an end view of the scalpel of FIG. 11;

FIGS. 13 and 14 are isometric and end views of the scalpel of FIGS. 11 and 12 to which has been added an inner housing;

FIGS. 15 and 16 are isometric and end views of the scalpel and inner housing of FIGS. 13 and 14 to which has been added a slidable blade guard;

FIGS. 17 and 18 are isometric and end views of the scalpel, inner housing and blade guard of FIGS. 15 and 16 to which has been added an outer housing, illustrating the outer housing in an retracted position exposing the scalpel blade;

FIG. 19 is an isometric view similar to FIG. 17 showing the blade guard in an extended or shielding position covering the scalpel blade;

FIG. 20 is an isometric view of a slightly different prior art flat scalpel handle;

FIGS. 21-23 are isometric views of a two piece blade protector illustrated as if in successive stages of assembly on the prior art handle of FIG. 20;

FIG. 24 is an enlarged cross-sectional view of the blade protector of FIGS. 21-23, taken substantially long line 24-24 of FIG. 22;

FIG. 25 is an enlarged partial broken view of the blade protector of FIGS. 21-23, illustrating a latch half on the blade guard;

FIG. 26 is an isometric view of a slightly different prior art hexagonal scalpel handle;

FIGS. 27-29 are isometric view of a two piece blade protector illustrated as if in successive stages of assembly on the prior art handle of FIG. 26;

FIG. 30 is a cross-sectional view of the inner housing of the embodiment of FIGS. 27-29, taken substantially along line 30-30 of FIG. 28 as viewed in the direction indicated by the arrows;

FIGS. 31-32 are end views of FIGS. 27-28 respectively;

FIG. 33 is a broken view of the end of the blade protector of FIGS. 27-29, taken substantially along line 33-33; and

FIG. 34 is a broken view of the end of the blade guard of FIGS. 28 and 29 showing one of the detents.

### DETAILED DESCRIPTION

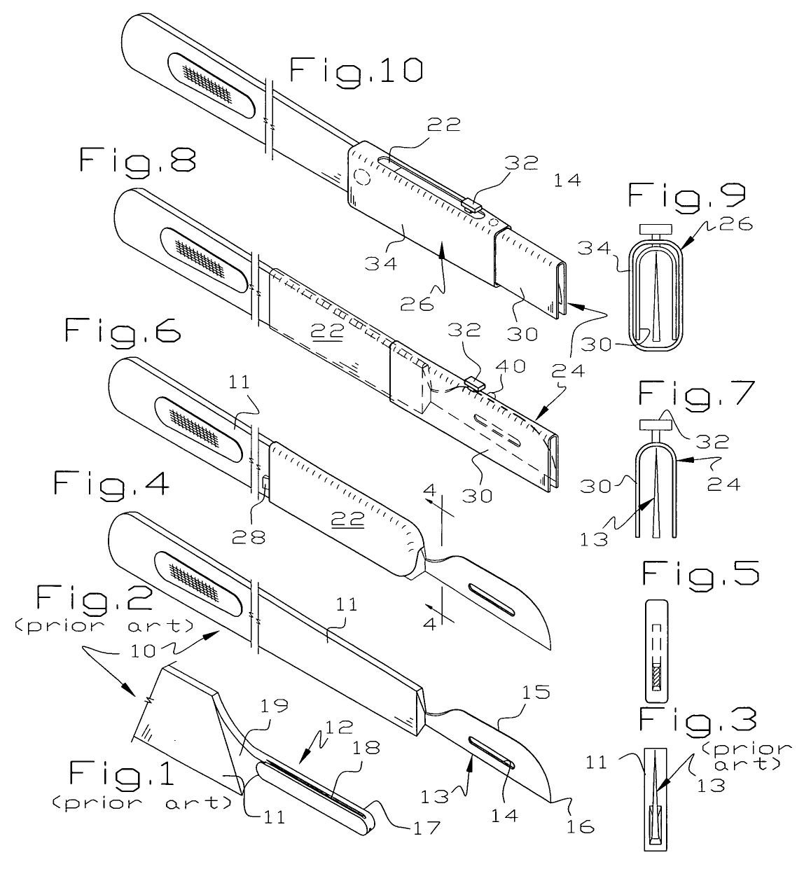

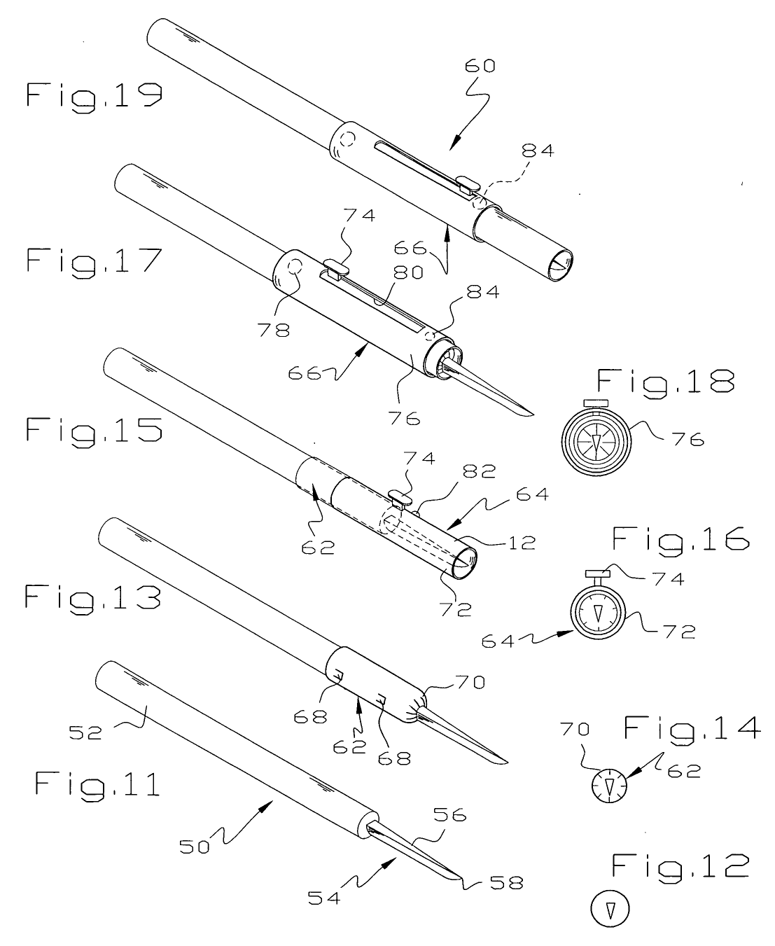

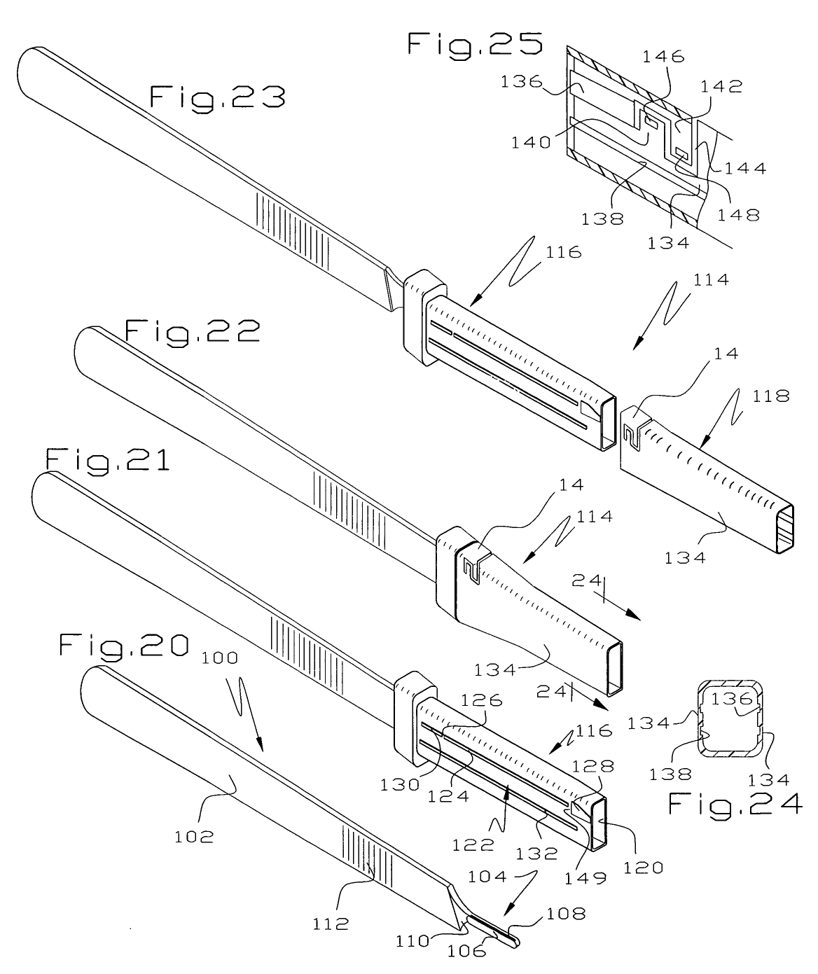

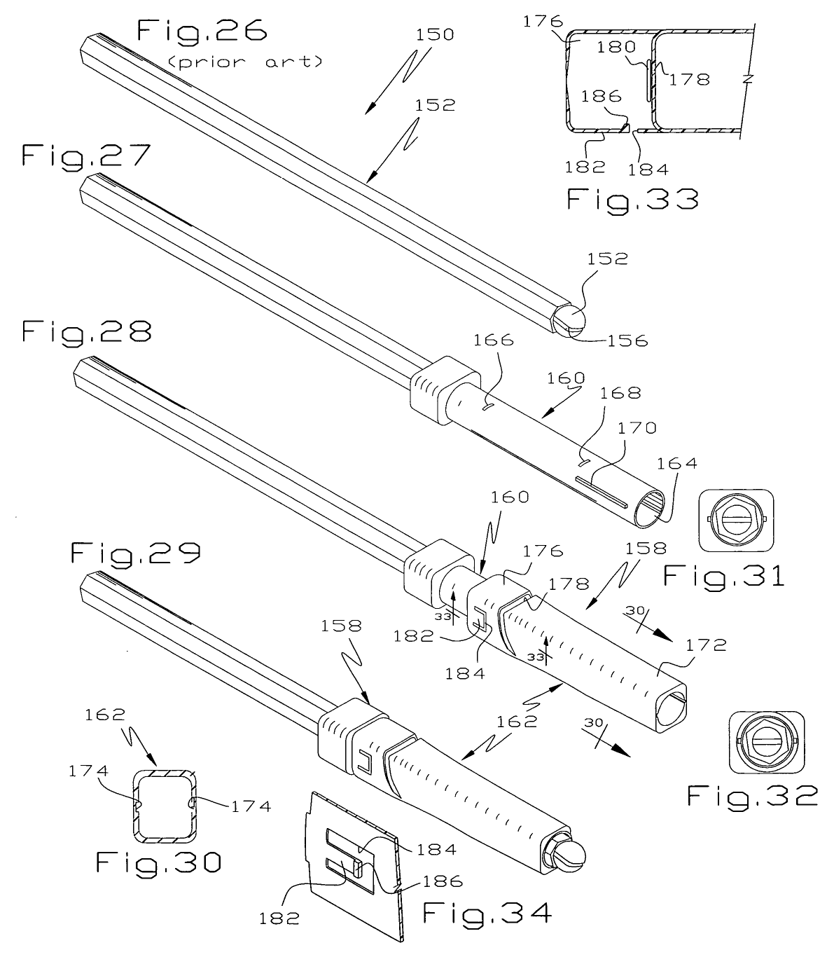

Referring to FIGS. 1-10, there is illustrated a conventional scalpel 10 having a flat handle 11 having an end 12 sculpted to receive a blade 13 having a slot 14 therein, a cutting edge 15 and a cutting tip 16. The end 12 provides an elongate rib 17 providing a notch 18 extending lengthwise and an inclined abutment 19. The rib 17, notch 18 and abutment 19 are sized and shaped to receive the slot 14 of the blade 13 and, in cooperation with the remainder of the blade 13, thereby attach the blade 13 to the handle 11. Provided on the scalpel 10 is a blade protector 20 comprising an inner housing 22, a blade guard 24 and an outer housing 26. As will become more fully apparent hereinafter, an important advantage of this invention is the ability to replace the blade 13 without removing the blade protector 20. Although FIGS. 2-9 appear to show the protected scalpel in the process of being assembled, this is for illustrative purposes only. The blade protector 20 is a unit and is attached in one motion.

The inner housing 22 is attached to the handle 11 in any suitable manner, such as by a friction fit. To this end, the inner housing 22 may be slightly elastic allowing it to be slipped over the end of the handle 11, after removing the blade 13. In the event the inner housing 22 does not snugly fit the scalpel handle 11, a wedge 28 or other suitable member may be inserted between the handle 11 and inner housing 22, as shown in FIG. 4. The inner housing 22 may be made of any suitable material, such as an injection molded medically approved polymer, and is mainly open so the blade 13 may be secured to the handle 11 after the blade protector 20 is in place. The inner housing 22 is smooth on the inside and free of any latch halves or mechanical devices to attach the inner housing 22 to a blade protector.

In the alternative, the inner housing 22 may be secured to the handle 11 by the use of releasable adhesives, by a shrink fit or the like. In the event a shrink fit is selected, the material of the inner housing 22 is selected to shrink in response to the application of heat, as with a hair dryer or the like. After use, and before sterilizing the handle 11, the blade protector 20 may be removed simply by cutting the material of the inner housing 22.

The blade guard 24 slides on the outside of the inner housing 22 between a position exposing the blade 13 and a position shielding the blade 13, as may be seen by a comparison of FIGS. 8 and 10. The blade guard 24 is made of any suitable material, such as an injection molded medically approved polymer, and preferably includes a U-shaped guard 30 having an opening adjacent the non-cutting edge of the blade 13 and an operator 32 conveniently placed for the surgeon to push or pull in order to slide the blade guard 24 between its positions. It will, of course, be evident that the blade guard 24 may be closed and the operator 32 placed on the top of the handle 11 rather than on the bottom as shown.

The outer housing 26 is made of any suitable material, such as an injection molded medically approved polymer, and includes a closed generally rectangular member 34 secured to the inner housing 22 in a suitable manner, as by pressure welding in a spot or depression 36 rearwardly of the blade guard 34. The member 34 provides a slot 38 along its edge so the operator 32 is exposed through the outer housing 26. It will accordingly be seen that rearward movement of the blade guard 24 is limited by the physical connection between the inner and outer housings 22, 26.

The position of the blade guard 24 relative to the inner and outer housings 22, 26 may be controlled in any suitable manner. Because the blade guard 24 slides between the inner and outer housings, the position control may be friction between the blade guard 24 and the housings 22, 26. In other words, the blade guard 24 slides, but not readily, between the housings 22, 26. In the alternative, suitable locking means, such as a detent or bulge 40 on the blade guard 24 and one or more depressions 42 facing the detent 40. The detent 40 and depression 42, or other similar arrangement, may also provide a limit of outward movement of the blade guard 24 so it cannot accidentally be advanced off the handle 11.

Use of the scalpel 10 and blade protector 20 should now be apparent. The blade protector 20 slips over the end of the scalpel handle 11 and the blade 13 is then installed. To position the blade guard 24 in the shielding position around the blade 13, the operator 32 is advanced to a position covering the tip 16 of the blade 13, as shown in FIG. 8. To use the scalpel 10, the surgeon manipulates the operator 32 to retract the blade guard 24 thereby exposing the cutting edge 15 and tip 16 of the blade 13 as shown in FIG. 10. It will also be seen that the blade 13 may be removed from the handle 11 with the blade protector 20 in place. This is of significant advantage because surgeons periodically replace blades during surgery and are accustomed to continue using the same handle.

Referring to FIGS. 11-19, there is illustrated another embodiment of a conventional scalpel 50 having a round/hexagonal handle 52 and a blade 54 providing a cutting edge 56 and a cutting tip 58. Provided on the scalpel 50 is a blade protector 60 comprising an inner housing 62, a blade guard 64 and an outer housing 66. Although FIGS. 11-17 appear to show the protected or guarded scalpel 50 in the process of being assembled, this is for illustrative purposes only. The blade protector 60 is a unit and is attached in one motion.

The inner housing 62 is attached to the handle 52 in any suitable manner, such as by a friction fit, by the use of releasable adhesives or by a shrink fit. To this end, the inner housing 52 may be slightly elastic allowing it to be slipped over the end of the handle 52. To insure that the inner housing 62 snugly fits the scalpel handle 52, one or more flaps 68 may be stamped in a side of the housing 62 to abut the handle 52 and thereby increase friction between the housing 62 and handle 52. The inner housing 62 may be made of any suitable material, such as an injection molded medically approved polymer, and includes a partially closed forward end 70 allowing the blade 54 to pass therethrough and preventing the inner housing 52 from moving too far rearwardly on the handle 52.

The blade guard 64 slides on the outside of the inner housing 52 between a position exposing the blade 54 and a position shielding the blade 54, as may be seen by a comparison of FIGS. 17 and 19. The blade guard 64 is made of any suitable material, such as an injection molded medically approved polymer, and preferably includes a circular guard 72 and an operator 74 conveniently placed for the surgeon to push or pull in order to slide the blade guard 64 between its positions. It will, of course, be evident that the blade guard 64 may be closed and the operator 74 placed on the top of the handle 52 rather than on the bottom as shown.

The outer housing 66 is made of any suitable material, such as an injection molded medically approved polymer, and includes a closed circular member 76 secured to the inner housing 52 in a suitable manner, as by pressure welding in a spot or depression 78 rearwardly of the blade guard 64. The member 78 provides a slot 80 extending lengthwise along the handle 52 so the operator 74 is exposed through the outer housing 66. It will accordingly be seen that rearward movement of the blade guard 64 is limited by the physical connection between the inner and outer housings 62, 66.

The position of the blade guard 64 relative to the inner or outer housings 62, 66 may be controlled in any suitable manner. Because the blade guard 64 slides between the inner and outer housings, the position control may be friction between the blade guard 64 and the housings 62, 66. In other words, the blade guard 64 slides, but not readily, between the housings 62, 66. In the alternative, suitable locking means, such as a detent or bulge 82 on the blade guard 64 and one or more depressions 84 facing the detent 82. The detent 82 and depression 84, or other similar arrangement, may also provide a limit of outward movement of the blade guard 64 so it cannot accidentally be advanced off the handle 52.

Use of the scalpel 50 and blade protector 60 should now be apparent. The blade protector 60 slips over the end of the scalpel handle 52 and the blade 54 is then installed. To position the blade guard 64 in the shielding position around the blade 13, the operator 74 is advanced to a position covering the tip 58 of the blade 54, as shown in FIG. 19. To use the scalpel 50, the surgeon manipulates the operator 74 to retract the blade guard 64 thereby exposing the cutting edge 56 and tip 58 of the blade 54 as shown in FIG. 17.

Referring to FIGS. 20-25, there is illustrated a conventional scalpel 100 having a simple unadorned flat handle 102 having an end 104 having an elongate rib 106 providing a notch 108 extending lengthwise and an inclined abutment 110. The rib 106, notch 108 and abutment 110 are sized and shaped to receive the slot of a conventional blade (not shown) and, in cooperation with the remainder of the blade, thereby attach the blade to the handle 102. The handle end 104 cooperates with a conventional blade to hold it securely in place. The handle 102 may include a series of vertical ribs 112 or other conventional grip enhancers but is free of any latches or mechanical devices to attach to a blade protector.

Provided on the scalpel 100 is a blade protector 114 comprising an inner housing 116 and an outer housing or blade guard 118. Although FIGS. 21-23 appear to show the protected scalpel in the process of being assembled, this is for illustrative purposes only. In some embodiments, the blade protector 108 is a unit and is attached in one motion.

The inner housing 116 is attached to the handle 102 by a friction fit, one species of which is a shrink fit. To this end, the inner housing 116 may be slightly elastic allowing it to be slipped over the end of the handle 102 either before or after removing the blade. In the event the inner housing 116 does not snugly fit the scalpel handle 102, a wedge (not shown) or other suitable member may be inserted between the handle 102 and inner housing 116. The inner housing 116 may be made of any suitable material, such as an injection molded medically approved polymer.

In the event a shrink fit is selected, the material of the inner housing 116 is selected to shrink in response to the application of heat, as with a hair dryer or the like. After use, and before sterilizing the handle 102, the blade protector 114 may be removed simply by cutting the material of the inner housing 116. The inner housing 116 is free of mechanical latches or coupling elements on the inside surface 120 and, in some embodiments, is smooth.

The outside of the inner housing 116 includes a latch half 122. In the embodiment of FIGS. 21-26, the latch half 122 includes a rail 124 having a gap 126 therein and an abutment 128 spaced from one end of the rail 124. Although the rail end 130 and the abutment 128 look different, their function is similar, i.e. to provide a gap for cooperation with a detent on the blade guard 114 as will be apparent hereinafter. In some embodiments, a second rail can be provided to facilitate linear movement of the blade guard 114 on the inner housing 116. It will shortly be apparent that the abutments 128, 130 act as a latch half. In some embodiments, a second rib 132 can be provided to aid in guiding the blade guard 114 during movement on the inner housing 116.

The blade guard 118 slides on the outside of the inner housing 116 between a position exposing the blade and a position shielding the blade, as may be seen by a comparison of FIGS. 22 and 23. The blade guard 118 is made of any suitable material, such as an injection molded medically approved polymer, and includes a box shaped body having side walls 134 providing a latch half on the inside as shown best in FIG. 25. The inside of the blade guard 118 includes grooves 136, 138 which straddle the ribs 124, 136 and guide the blade protector 118 for movement on the inner housing 116.

In some embodiments, the latch half on the inside of the blade protector 118 includes first and second pairs of arms 140, 142 which are basically coplanar with the walls 134 and formed by a slot 144 in the walls 134. The arms 140, 142 are accordingly connected to the side walls 134 by a live hinge at the base of the arms 140, 142, a live hinge being a resilient connection between a free end of each arm and the side wall 134. The slot 144 also allows movement of the arms 140, 142 because the arm base is part of the side wall 134 while the free end of the arms 140, 142 are capable of movement. The first pair of arms 140 include a detent or projection 146 positioned to enter the gap 126 and thereby latch the blade protector 118 in the retracted position on the inner housing 116. The second pair of arms 142 includes a detent or projection 148 positioned to abut the shoulder 128 and prevent the blade guard 118 from passing off the end of the inner housing 116. FIG. 25 reveals there is a mirror image set of arms 140, 142 on each side of the blade guard 118.

The position of the blade guard 118 relative to the inner housing 116 may be controlled in any suitable manner. Because the blade guard 118 slides on the inner housing, the position control may be friction between the blade guard 118 and the housing 116 but the end positions are controlled by the detents 146, 148 on the inside of the blade guard 118 and the gaps in the rib 124 on the outside of the inner housing 116.

Use of the scalpel 100 and blade protector 114 should now be apparent. The blade protector 114 slips over the end of the scalpel handle 102 and the blade is then installed. To position the blade guard 114 in the shielding position around the blade, the blade guard 118 is advanced to a position covering the end of the blade at a location where the detent 148 abuts the shoulder 128 thereby preventing the blade guard 114 from sliding off the end of the inner housing 116. This is accomplished by pushing down on the pad 147 which flexes the end of the blade guard 114 and moving the detent 146 out of the gap 126. Then, the blade guard 114 slides easily on the inner housing 116. In some embodiments, the blade guard 114 may simply be pulled so the detent 146 cams its way out of the gap 126.

To use the scalpel 100, the surgeon retracts the blade guard 118. This is accomplished by pushing down on the pad 147 and pulling on the blade guard 118. Pushing down on the pad 147 moves the detent 148 laterally away from its position between the end of the rib 124 and the shoulder 128. In other words, pushing down on the pad 147 flexes the end of the blade guard 118 and moves the detent 148 out of the gap 149. Pulling on the blade guard 118 retracts it onto the inner housing 116 thereby exposing the blade on the end of the handle end 104. It will be seen that the blade may be removed from the handle 102 with the blade protector 114 in place. This is of significant advantage because surgeons periodically replace blades during surgery and are accustomed to continue using the same handle.

Referring to FIGS. 26-34, there is illustrated another embodiment of a conventional scalpel 150 having a round/hexagonal handle 152 providing an end 154 having a slot 156 for receiving a blade (not shown). Provided on the scalpel 150 is a blade protector 158 comprising an inner housing 160 and a blade guard 162. Although FIGS. 26-29 appear to show the protected or guarded scalpel 150 in the process of being assembled, this is for illustrative purposes only. The blade protector 158 is a unit and is attached in one motion.

The inner housing 160 provides a circular or polygonal passage 164 therethrough attached to the handle 152 by a friction fit, one species of which is a shrink fit. To this end, the inner housing 160 may be slightly elastic allowing it to be slipped over the end of the handle 152 either before or after removing the blade. In the event the inner housing 160 does not snugly fit the scalpel handle 152, a wedge (not shown) or other suitable member may be inserted between the handle 152 and inner housing 160. The inner housing 160 may be made of any suitable material, such as an injection medically approved polymer.

In the event a shrink fit is selected, the material of the inner housing 160 is selected to shrink in response to the application of heat, as with a hair dryer or the like. After use, and before sterilizing the handle 152, the blade protector 158 may be removed simply by cutting the material of the inner housing 160. The inner housing 160 is free of mechanical latches or coupling elements on the inside surface 164 and, in some embodiments, is smooth. The outside of the inner housing 160 includes a latch half. In some embodiments, the latch half includes a pair of notches or recesses 166, 168 and a rib 170 on opposite sides of the inner housing 160 for guiding the blade guard 162.

The blade guard 162 slides on the outside of the inner housing 160 between a position exposing the blade and a position shielding the blade, as may be seen by a comparison of FIGS. 28 and 29. The blade guard 162 is made of any suitable material, such as an injection molded medically approved polymer, and in some embodiments includes a more-or-less circular body 172 providing a latch half on the inside as shown best in FIG. 34. The inside of the blade guard 162 includes grooves 174 which receive the rib 170 and guide the blade protector 158 for movement on the inner housing 160.

In some embodiments, the latch half on the inside of the blade protector 162 includes a single arm or pad 176 which is formed by a slot 178 in the blade guard 162. A detent 180 on the arm 176 is positioned to be received in one of the recesses 166, 168 and thereby latch the blade protector 162 in the retracted position or in the extended position on the inner housing 160. In some embodiments, the blade protector 162 includes a second arm 182 formed by a slot 184. The arm 182 includes a detent or projection 186 which abuts an end of the rib 170 in the extended position of the blade guard 162 and, in conjunction with the detent 180 and recess 168, prevents the blade guard 162 from passing off the end of the inner housing 160. When the blade guard 162 is pushed onto the inner housing 160, the arm 162 flexes thereby allowing the detent 186 to clear the rib 170. In this version, the arm 182 and detent 186 act only to prevent the blade guard 162 from sliding off the end of the inner housing 60. The arms 176, 182 are accordingly connected to the wall of the body 172 by a live hinge at the base of the arms 176, 182, a live hinge being a resilient connection between a free end of each arm and the body 172.

The position of the blade guard 162 relative to the inner housing 160 may be controlled in any suitable manner. Because the blade guard 162 slides on the inner housing 160, the position control may be friction between the blade guard 162 and the housing 160 but the end positions are controlled by the detents 180, 186 on the inside of the blade guard 162 and the abutments they encounter on the inside of the inner housing 160.

Use of the scalpel 150 and blade protector 158 should now be apparent. The blade protector 158 slips over the end of the scalpel handle 152 and the blade is then installed. To position the blade guard 162 in the shielding position around the blade, the blade guard 162 is advanced to a position covering the end of the blade at a location where the detent 180 comes to rest in the recess 168 and/or the detent 186 abuts the end of the rib 170. This is accomplished by pushing down on the arm 176 which flexes the end of the blade guard 162 and moving the detent 180 out of the notch 166.

Then, the blade guard 162 slides easily on the inner housing 160. In some embodiments, the blade guard 162 may simply be pulled so the detent 178 cams its way out of the recess 166.

To use the scalpel 150, the surgeon retracts the blade guard 162. This is accomplished by pushing down on the arm 176 and pulling on the blade guard 162. Pushing down on the pad 176 moves the detent 180 out of the recess 166. In other words, pushing down on the arm 176 flexes the end of the blade guard 162 and moves the detent 180 out of the recess 166. Pulling on the blade guard 162 retracts it onto the inner housing 160 thereby exposing the blade on the end of the handle end 152. It will be seen that the blade may be removed from the handle 152 with the blade protector 158 in place. This is of significant advantage because surgeons periodically replace blades during surgery and are accustomed to continue using the same handle.

Although this invention has been disclosed and described in its preferred forms with a certain degree of particularity, it is understood that the present disclosure of the preferred forms is only by way of example and that numerous changes in the combination and arrangement of parts may be made without departing from the spirit and scope of the invention as hereinafter claimed.

I claim:

1. A scalpel comprising a handle having a receiver on an end of the handle for receiving a blade providing a cutting edge, and a blade protector including an inner housing having an inner surface free of latching mechanisms frictionally fit onto the handle, a blade guard mounted on the inner housing and slidable between a retracted position for exposing the blade edge and an extended position for shrouding the blade edge and a latch mechanism for securing the blade guard to the inner housing in the retracted position and for securing the blade guard to the inner housing in the extended position.

2. The scalpel of claim 1 wherein the blade guard includes an inner passage receiving the inner housing, the inner passage being smooth.

3. The scalpel of claim 1 wherein the handle is flat.

4. The scalpel of claim 1 wherein the handle is round.

5. The scalpel of claim 1 wherein the handle is hexagonal.

6. The scalpel of claim 1 wherein the latch mechanism includes recesses on an exterior of the inner housing and a movable arm on an interior of the blade guard, the movable arm having a detent for engaging one of the recesses on the inner housing.

7. The scalpel of claim 6 wherein the blade guard comprises a wall surrounding a passage therethrough, the wall having a slot outlining the arm and providing a live hinge allowing a free end of the arm to move.

8. The scalpel of claim 1 wherein
the inner housing provides a first recess adjacent a proximal end of the inner housing and a second recess adjacent a distal end of the inner housing, and
the blade guard comprises a wall surrounding a passage therethrough, the wall having a slot outlining an arm having a detent thereon and providing a live hinge allowing a free end of the arm to move into engagement with the recess for securing the blade guard to the inner housing in the retracted position and allowing the free end of the arm to move into engagement with the second recess for securing the blade guard to the inner housing in the extended position.

9. The scalpel of claim 1 wherein
the inner housing provides a first recess adjacent a proximal end of the inner housing and a second recess adjacent a distal end of the inner housing, and
the blade guard comprises a wall surrounding a passage therethrough, the wall having
a first slot outlining a first arm having a first detent on a free end of the first arm and providing a first live hinge allowing a free end of the first arm to move into engagement with the first recess for securing the blade guard to the inner housing in the retracted position, and
a second slot outlining a second arm having a second detent on a free end of the second arm and providing a second live hinge allowing a free end of the second arm to move into engagement with the second recess for securing the blade guard to the inner housing in the extended position.

10. The blade protector of claim 1 wherein the blade guard comprises an exterior of the blade protector.

11. A blade protector for a scalpel comprising a handle having a receiver on an end of the handle for receiving a blade providing a cutting edge comprising a blade protector including an inner housing having an inner surface free of latching mechanisms for frictionally fitting onto the handle, a blade guard mounted on the inner housing and slidable between a retracted position for exposing the blade edge and an extended position for shrouding the blade edge and a latch mechanism for securing the blade guard in the retracted position to the inner housing and for securing the blade guard to the inner housing in the extended position.

12. The scalpel of claim 11 wherein the blade guard includes an inner passage receiving the inner housing, the inner passage being smooth.

13. The scalpel of claim 11 wherein the latch mechanism includes recesses on an exterior of the inner housing and a movable arm on an interior of the blade guard, the movable arm having a detent for engaging one of the recesses on the inner housing.

14. The scalpel of claim 13 wherein the blade guard comprises a wall surrounding a passage therethrough, the wall having a slot outlining the arm and providing a live hinge allowing a free end of the arm to move.

15. The scalpel of claim 11 wherein
the inner housing provides a first recess adjacent a proximal end of the inner housing and a second recess adjacent a distal end of the inner housing, and
the blade guard comprises a wall surrounding a passage therethrough, the wall having a slot outlining an arm having a detent thereon and providing a live hinge allowing a free end of the arm to move into engagement with the recess for securing the blade guard to the inner housing in the retracted position and allowing the free end of the arm to move into engagement with the second recess for securing the blade guard to the inner housing in the extended position.

16. The scalpel of claim 11 wherein
the inner housing provides a first recess adjacent a proximal end of the inner housing and a second recess adjacent a distal end of the inner housing, and
the blade guard comprises a wall surrounding a passage therethrough, the wall having
a first slot outlining a first arm having a first detent on a free end of the first arm and providing a first live hinge allowing a free end of the first arm to move into engagement with the first recess for securing the blade guard to the inner housing in the retracted position, and
a second slot outlining a second arm having a second detent on a free end of the second arm and providing a second live hinge allowing a free end of the second arm to move into engagement with the second recess for securing the blade guard to the inner housing in the extended position.

17. The scalpel of claim 11 wherein the blade guard comprises an exterior of the blade protector.
